SEQUENCE LISTING (8 sequences)

SEQ ID NO: 1 - DNA, 21 bp, synthetic construct
  source 1..21: mol_type = other DNA
tggagttgatggatgcttgag

SEQ ID NO: 2 - DNA, 20 bp, synthetic construct
  source 1..20: mol_type = other DNA
ggacactggagatgcttgat

SEQ ID NO: 3 - DNA, 20 bp, synthetic construct
  source 1..20: mol_type = other DNA
aaggctgcaagggctttttc

SEQ ID NO: 4 - DNA, 20 bp, synthetic construct
  source 1..20: mol_type = other DNA
ctgcgtacaagctgtctctt

SEQ ID NO: 5 - DNA, 21 bp, synthetic construct
  source 1..21: mol_type = other DNA
cgtcctcactttcaacttcca

SEQ ID NO: 6 - DNA, 21 bp, synthetic construct
  source 1..21: mol_type = other DNA
gacacgacatagctgacaaga

SEQ ID NO: 7 - DNA, 20 bp, synthetic construct
  source 1..20: mol_type = other DNA
ggagaaagctgccaaatatg

SEQ ID NO: 8 - DNA, 20 bp, synthetic construct
  source 1..20: mol_type = other DNA
accaggaaatgagcttgaca

(19) United States
(12) Patent Application Publication
(10) Pub. No.: US 2025/0388970 A1
(43) Pub. Date: Dec. 25, 2025

(54) Treatment of Atopic Dermatitis Using Mesenchymal Stem Cells and Immune Modulation

(71) Applicant: PrimeGen Biotech, LLC, Santa Ana, CA (US)

(72) Inventors: Fariborz Izadyar, Santa Ana, CA (US); Chad Maki, Santa Ana, CA (US); Thomas Ramos, Santa Ana, CA (US)

(21) Appl. No.: 19/194,850

(22) Filed: Apr. 30, 2025

Related U.S. Application Data
(62) Division of application No. 17/426,576, filed on Jul. 28, 2021.
(60) Provisional application No. 62/799,300, filed on Jan. 31, 2019.

(51) Int. Cl. C12Q 1/6883

(52) U.S. Cl. CPC C12Q 1/6883; C12Q 2600/112; C12Q 2600/118; C12Q 2600/158; C12Q 2600/178

(57) ABSTRACT

Provided are methods of diagnosis and treatment of atopic dermatitis.

### RELATED APPLICATIONS

This application is a divisional of U.S. patent application Ser. No. 17/426,576, filed Jul. 28, 2021, which is a 371 of PCT/US2020/016191, filed Jan. 31, 2020, which claims the benefit of U.S. Provisional Patent Application Ser. No. 62/799,300, filed Jan. 31, 2019, the contents of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 16, 2025, is named 19194850_SL.xml and is 8,094 bytes in size.

### TECHNICAL FIELD

This disclosure relates to methods and compositions for diagnosis and treatment of inflammatory skin diseases using Mesenchymal Stem Cells.

### BACKGROUND

Canine atopic dermatitis (AD) is a genetically-predisposed inflammatory and pruritic allergic skin disorder that affects approximately 10% of dogs worldwide. Although pathogenesis of canine AD remains elusive, epidermal barrier dysfunction and immune dysregulation following allergen exposure are believed to be implicated in development of AD. It is also known that allergic skin inflammation is in part attributed to diminished skin barrier function and increased Type 2 Helper T Cell (Th2) activity. In the acute phase, defects in the skin barrier facilitate contact of the environmental allergens to epidermal antigen presenting cells (APCs). The APCs then capture the allergens and present them to IgE-coated mast cells which can release histamine, cytokines, and chemokines. A plethora of immune cells migrate into the vicinity, including eosinophils and Th2 cells. Th2 cells in turn secrete pro- and anti-inflammatory cytokines including IL-4, IL-13, IL-5, IL-31 and IL-10. After the acute Th2 response, it is thought that a subsequent Type 1 Helper T Cell (Th1) response occurs, mediated by factors including interferon-γ (IFN-γ).

To date, diagnosis of canine AD remains clinical examination and exclusion of other possible causes, and no reliable biomarkers are available to distinguish canine AD from other similarly presenting diseases. To address this issue, efforts have been made by examining specific immune cells, cytokines and genes in peripheral blood of both AD dogs and healthy controls. However, only limited studies with some contradictory results have been reported in this area.

### SUMMARY

Disclosed herein are methods for diagnosing atopic dermatitis (AD) comprising determining the expression levels of at least one marker, for example miR-203 or miR-483, and comparing said expression levels with those in a patient without AD, wherein increased miR-203 and/or miR-483 expression levels indicate a patient suffering from AD.

Further disclosed are methods for diagnosing AD comprising determining the expression levels of, for example, PIAS1, RORA, SH2B1 and comparing said expression levels with those in a patient without AD, wherein decreased PIAS1, RORA, or SH2B1 expression levels indicate a patient suffering from AD.

Further disclosed are methods for diagnosing AD comprising determining the expression level of, for example, phosphodiesterase 4D (PDE4D) gene in peripheral blood mononuclear cells (PBMCs), and comparing said expression levels with those in a patient without AD, wherein increased expression levels indicate a patient suffering from AD.

Further disclosed are methods for pre-selecting AD patients to be appropriate for adipose-derived mesenchymal stem cell (MSC) treatment, wherein said pre-selecting comprises determining the expression levels of at least one marker, for example miR-203 and miR-483, and comparing said expression levels with those in a patient without AD, wherein increased miR-203 and miR-483 expression levels indicate a patient suffering from AD, or determining the expression levels of, for example, PIAS1, RORA, SH2B1 and comparing said expression levels with those in a patient without AD, wherein decreased PIAS1, RORA, SH2B1 expression levels indicate a patient suffering from AD, or determining the expression level of, for example, phosphodiesterase 4D (PDE4D) gene in peripheral blood mononuclear cells (PBMCs) and comparing said expression levels with those in a patient without AD, wherein increased expression levels indicate a patient suffering from AD.

Further disclosed are methods to correlate MSC potency by testing methods, for example methods for diagnosing atopic dermatitis (AD) comprising determining the expression levels of, for example, miR-203 and miR-483, and comparing said expression levels with those in a patient without AD, wherein increased miR-203 and miR-483 expression levels indicate a patient suffering from AD, methods for diagnosing AD comprising determining the expression levels of PIAS1, RORA, SH2B1 and comparing said expression levels with those in a patient without AD, wherein decreased PIAS1, RORA, SH2B1 expression levels indicate a patient suffering from AD, and methods for diagnosing AD comprising determining the expression level of phosphodiesterase 4D (PDE4D) gene in peripheral blood mononuclear cells (PBMCs) and comparing said expression levels with those in a patient without AD, wherein increased expression levels indicate a patient suffering from AD, for AD patient screening and improvement post-treatment.

Further disclosed are methods for treating AD comprising administration of MSC, for example modified or stimulated MSC, to a patient in need thereof. Further disclosed are methods wherein said patient is a mammal, particularly canine and human.

Further disclosed are methods wherein said MSC is obtained from adipose tissue, bone marrow, umbilical cord or placenta. Further disclosed are methods wherein said administration comprises at least one of subcutaneous, intra-articular, intra-lesional, intravenous, intra-peritoneal or intramuscular administration. Further disclosed are methods wherein MSCs are administered 1-10 times with 1-6 months intervals.

Further disclosed are methods wherein said MSC are autologous. Further disclosed are methods wherein said MSC are allogenic. Further disclosed are methods wherein said MSC are administered in a dose between 1×10³ cells and 1×10¹² cells.

Further disclosed are methods for modifying MSC to produce a cytokine, comprising altering the genetic makeup of the MSC, wherein altering the genetic makeup of the MSC can comprise introduction of non-native DNA or stimulation of expression of native DNA, or both.

Further disclosed are methods for stimulating MSC to produce a cytokine, for comprising applying a signaling molecule the MSC, wherein the signaling molecule can comprise, for example, a cytokine, mRNA, miRNA, or the like.

In embodiments, the immune system of atopic dermatitis patient is imbalanced and has an abnormal CD4:CD8 ratio.

In embodiments, mesenchymal stem cells are stimulated by one, two or more cytokines prior administration. In embodiments, MSCs will be incubated with other factors selected from at least one atopic dermatitis biomarker. In embodiments, The stimulants can be added all at the same time or in different orders, for example, sequentially, to achieve maximum effect.

In embodiments, cytokines and biomarkers are chosen by comparing the blood of the normal control patients and the blood of the patients with atopic dermatitis.

In embodiments, MSCs must be incubated with stimulatory cytokines or biomarkers for a minimum of 12 h and a maximum of 24 h.

In embodiments, after co incubation of MSCs with cytokines or other factors, the cells are washed to remove excess stimulants.

In embodiments, cytokines used for MSC stimulation will result in production of other cytokines by the MSCs that modulate the immune system of the patient systemically and locally at the skin site.

In embodiments, MSCs can migrate to the site of inflammation at the skin and directly interact with the immune cells resident at the site of skin inflammation.

In embodiments, stimulated MSCs have an accelerated effect on immune balance of the host result in quicker CD4:CD8 balance.

In embodiments, MSCs can be modified by genetic manipulation to become more anti allergic.

In embodiments, modification of MSCs can be achieved by insertion of cDNA for upregulation of a factor that is anti-allergic or by downregulation of factors that are allergy inducers through miRNA or knock-out technique.

Further disclosed is a composition comprising (a) isolated mesenchymal stem cells; (b) isolated interferon gamma; and (c) isolated interleukin-1 alpha, interleukin-1 beta or tumor necrosis factor alpha, in admixture with a pharmaceutically acceptable carrier. A kit for attenuating an immune response is also provided.

Further disclosed is a method for attenuating an immune response by administering an effective amount of a disclosed composition to a subject in need of treatment.

Further disclosed are methods for enhancing a local immune response is also provided. This method involves administering to a subject in need of treatment an effective amount of iNOS-deficient or IDO-deficient mesenchymal stem cells thereby enhancing a local immune response. In certain embodiments, the local immune response is to a vaccine or tumor.

### FIGURES

FIG. 1. RT-PCR results show elevated expression of PDE4 gene AD dogs compared to the healthy controls. Expression levels of PDE4A (A), PDE4B (B) and PDE4D (C) were elevated in the PBMCs of canine AD dogs in comparison with those of the healthy controls. Canine GAPDH gene was used as an internal control. Each bar is representative of a triplicate experiment for each patient (healthy: n=8, Atopic: n=9). T-test; ns—Not Significant, * P<0.05, ** P<0.01. RT-PCR results represent relative expression of AD dogs normalized to that of the health controls.

FIG. 2. RT-PCR results show elevated expression of miR-203 and miR-483 and decreased expression of the specific genes (PIAS1, RORA and SH2B1) in canine AD dogs compared to the healthy controls. (A) Expression levels of miR-203 and miR-483 were elevated in the plasma of canine AD dogs by approximately 2.5-fold and 1.6-fold respectively in comparison with those of the healthy controls. The canine miR-39 was used as the internal control. (B) Expression levels of the three specific genes (PIAS1, RORA and SH2B1) were significantly downregulated in PBMCs of the canine AD dogs compared to the healthy dogs. Canine GAPDH gene was used as an internal control. Each bar is representative of a triplicate experiment for each patient (Healthy: n=8, Atopic: n=9). T-test; ns—Not Significant, * P<0.05, ** P<0.01. RT-PCR results represent relative expression of AD dogs normalized to that of the health controls.

FIG. 3. Analysis of CD4⁺ T Cell compared to CD8⁺ T Cells in healthy vs Atopic Canines. (A) CD4 vs CD8 flow plot from PBMCs of 1 healthy canine and 1 atopic canine. All plots were gated on lymphocytes and CD3⁺ Cells. Dead cells were excluded by 7-AAD. (B) Comparison of CD4⁺/CD8⁺ T Cell ratios between healthy canines (n=8) and atopic canines (n=9). The mean CD4⁺/CD8⁺ ratio of healthy canines was 2.031±0.3105 compared to 2.21±0.3626 for atopic canines (results expressed as mean ratio±SEM). P=0.3585 and not significant.

FIG. 4. Cytokine Profiles of Healthy vs Atopic Canines. Serum was isolated from whole blood extracted from either healthy or AD dogs. Expression of a multitude of cytokines including A) IL-13, B) IL-31, C) TNF-α, D) IFN-γ, E) IL-10, F) IL-4, G) TGF-β1 were analyzed via ELISA. Each bar is representative of a duplicate experiment for each patient (Healthy: n=8, Atopic: n=9). T-test; NS—Not Significant, *P<0.05, ** P<0.01, *** P<0.001, **** P<0.001.

### DETAILED DESCRIPTION

As used herein, the term “about” will mean up to plus or minus 5% of the particular term.

As used herein, the phrase “consisting essentially of” refers to excluding other active ingredients or any other ingredient that can materially affect the basic characteristic of a composition, formulation or structure, but generally including excipients.

As used herein, an “effective amount” refers to that amount of stem cells, cytokines, or a therapeutic composition containing both, that is sufficient to modulate, attenuate, or induce an immune response (i.e., suppression of T cell responses or promotion of an immune response) in the subject thereby reducing at least one sign or symptom of the disease or disorder under treatment.

As used herein, the terms “treat,” “treating,” or “treatment” and the like refers to alleviating signs or symptoms of the disease accomplished by a administering a composition to a patient in need of such treatment. Such alleviation can occur prior to signs or symptoms of the disease appearing, as well as after their appearance, therefore it encompasses prophylactic and active treatment. In addition, “treat,” “treating” or “treatment” does not require complete alleviation of signs or symptoms, or a cure. At a cellular level it may include reduction of diseased or target cellular population by at least 10%, 25%, 50%, 75%, 80%, 85%, 90%, 95%, or 99% as compared to untreated cells or cells treated with control or a comparative agent.

As used herein, the terms “administration” or “administering” or “treatment regimen” within the scope of the present invention includes a single therapeutic delivery, or multiple or repeated deliveries, or a control delivery therapeutic of any of the individual components of the present invention or in combination. Such terms are further meant to include modes of deliveries such as locally, systemically, intravascularly, intramuscularly, intra-peritoneally, inside the blood-brain barrier, organ-specific interventional injection or via other various routes.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The term “treatment” or “treating” refers to any therapeutic intervention in a mammal, for example a companion animal, including: (i) prevention, that is, causing the clinical symptoms not to develop, e.g., preventing infection or inflammation from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection.

The terms “reducing”, “suppressing” and “inhibiting” have their commonly understood meaning of lessening or decreasing.

The terms “effective,” “effective amount,” and “therapeutically effective amount” refer to that amount of MSC and/or a pharmaceutical composition thereof that produces a beneficial result.

The phrases “parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “pharmaceutical composition” refers to a formulation containing the therapeutically active agents described herein in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., MSC) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. In a preferred embodiment, the active ingredients are mixed under sterile conditions with a pharmaceutically acceptable carrier, and with any preservatives, buffers, or propellants that are required.

The terms “pharmaceutically acceptable” or “therapeutically acceptable” refers to a substance which does not interfere with the effectiveness or the biological activity of the active ingredients and which is not toxic to the host.

The phrase “pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A “patient,” “subject,” or “host” to be treated by the subject method may mean either a human or non-human animal, such as a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The term “in vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

### Diagnosis of Atopic Dermatitis

Disclosed herein are methods for diagnosing AD. For example, in embodiments, disclosed are methods for diagnosing atopic dermatitis (AD) comprising determining the expression levels of at least one marker, for example miR-203 or miR-483, and comparing said expression levels with those in a patient without AD, wherein increased miR-203 and/or miR-483 expression levels indicate a patient suffering from AD.

Further disclosed are methods for diagnosing AD comprising determining the expression levels of, for example, PIAS1, RORA, SH2B1 and comparing said expression levels with those in a patient without AD, wherein decreased PIAS1, RORA, or SH2B1 expression levels indicate a patient suffering from AD.

Further disclosed are methods for diagnosing AD comprising determining the expression level of, for example, phosphodiesterase 4D (PDE4D) gene in peripheral blood mononuclear cells (PBMCs), and comparing said expression levels with those in a patient without AD, wherein increased expression levels indicate a patient suffering from AD.

Further disclosed are methods for pre-selecting AD patients to be appropriate for adipose-derived mesenchymal stem cell (MSC) treatment, wherein said pre-selecting comprises determining the expression levels of at least one marker, for example miR-203 and miR-483, and comparing said expression levels with those in a patient without AD, wherein increased miR-203 and miR-483 expression levels indicate a patient suffering from AD, or determining the expression levels of, for example, PIAS1, RORA, SH2B1 and comparing said expression levels with those in a patient without AD, wherein decreased PIAS1, RORA, SH2B1 expression levels indicate a patient suffering from AD, or determining the expression level of, for example, phosphodiesterase 4D (PDE4D) gene in peripheral blood mononuclear cells (PBMCs) and comparing said expression levels with those in a patient without AD, wherein increased expression levels indicate a patient suffering from AD.

Further disclosed are methods to correlate MSC potency by testing methods, for example methods for diagnosing atopic dermatitis (AD) comprising determining the expression levels of, for example, miR-203 and miR-483, and comparing said expression levels with those in a patient without AD, wherein increased miR-203 and miR-483 expression levels indicate a patient suffering from AD, methods for diagnosing AD comprising determining the expression levels of PIAS1, RORA, SH2B1 and comparing said expression levels with those in a patient without AD, wherein decreased PIAS1, RORA, SH2B1 expression levels indicate a patient suffering from AD, and methods for diagnosing AD comprising determining the expression level of phosphodiesterase 4D (PDE4D) gene in peripheral blood mononuclear cells (PBMCs) and comparing said expression levels with those in a patient without AD, wherein increased expression levels indicate a patient suffering from AD, for AD patient screening and improvement post-treatment.

### Treatments for Atopic Dermatitis

Disclosed herein are treatments for AD, in particular canine treatments, comprising administration of stem cells. In embodiments, the MSC are stimulated or modified to produce a cell signaling molecule, for example a cytokine.

Stem cells are specialized cells, capable of renewing themselves through cell division as well as differentiating into multi-lineage cells. These cells are categorized as embryonic stem cells (ESC), induced pluripotent stem cells (iPSC), and adult stem cells. Mesenchymal stem cells (MSC) are adult stem cells which can be isolated from human and animal sources. Human MSC (hMSC) are non-haematopoietic, multipotent stem cells with the capacity to differentiate into mesodermal lineage such as osteocytes, adipocytes and chondrocytes as well ectodermal (neurocytes) and endodermal lineages (hepatocytes). MSC express cell surface markers including cluster of differentiation (CD) 29, CD44, CD73, CD90, CD105, and lack the expression of CD14, CD34, CD45, and HLA (human leucocyte antigen)-DR. hMSC have been isolated from various tissues, including adipose tissue, amniotic fluid, endometrium, dental tissues, umbilical cord, and Wharton's jelly. hMSC have been cultured long-term in specific media without any severe abnormalities. Furthermore, MSC have immunomodulatory features, and can secrete cytokines and immune-receptors which regulate the microenvironment in the host tissue. Multilineage potential, immunomodulation and secretion of anti-inflammatory molecules makes MSC an effective tool in the treatment of chronic diseases. MSC are not to be confused with haematopoietic (blood) stem cells that are also found in bone marrow. Morphologically, mesenchymal stem cells have long thin cell bodies with a large nucleus. As with other stem cell types, MSC have a high capacity for self renewal while maintaining multipotency.

MSC are typically identified based upon the expression or lack of expression of particular markers. For example, MSCs are CD34−, CD1 1 b, CD11c−, CD45−, MHC class II, CD44+, Sca-1+, and MHC class I low. In addition, MSCs can be identified by their ability to differentiate into various mesenchymal cell types. In vitro experiments have demonstrated that culture conditions, additives, growth factors and cytokines can precisely induce MSC to develop into a selected mesenchymal cells. For example, dexamethasone in combination with isobutilmethylxanthine or insulin or a mixture of isobutilmethylxanthine, insulin and indomethacin has been shown to push the MSCs toward differentiating into adipocytes. Similarly, MSCs can differentiate into skeletal muscle cells when stimulated with 5-azacytidine. 13-VGF has been shown to cause mesenchymal stem cells to differentiate into cardiac muscle cells.

Disclosed embodiments comprise compositions for treating a patient, for example an animal such as a canine, suffering from an inflammatory disease such as atopic dermatitis, said composition comprising MSC derived from progenitor cells harvested from, for example, placental tissue, bone marrow, dental tissue, testicle tissue, uterine tissue, umbilical cord tissue, or skin tissue that are allogeneic or autologous to a target patient; and a saline solution, and wherein the composition is operable to reduce or eliminate the symptoms of the dermatitis. In embodiments, the MSC can be stimulated or modified, for example by introducing non-native DNA or applying a cell signaling molecule.

Embodiments comprise combination treatments comprising administration of stem cells with another active agent, for example a PDE4 (phosphodiesterase-4) inhibitor.

Further disclosed are methods for modifying MSC to produce a cytokine, comprising altering the genetic makeup of the MSC, wherein altering the genetic makeup of the MSC can comprise introduction of non-native DNA or stimulation of expression of native DNA, or both.

Further disclosed are methods for stimulating MSC to produce a cytokine, for comprising applying a signaling molecule the MSC, wherein the signaling molecule can comprise, for example, a cytokine, mRNA, miRNA, or the like.

In embodiments, isolated MSC can be formulated into a pharmaceutically-acceptable composition, for example by using at least one pharmaceutically-acceptable carrier. In embodiments, a pharmaceutically-acceptable carrier means a carrier that is useful in preparing a pharmaceutical composition or formulation that is generally safe, non-toxic, and neither biologically nor otherwise undesirable, and includes a carrier that is acceptable for veterinary use as well as human pharmaceutical use. The pharmaceutically acceptable carrier can comprise, for example, saline solution, phosphate buffered saline (PBS), Ringer's serum, Ringer's lactate serum, lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils.

Disclosed embodiments can comprise administration of MSC to treat atopic dermatitis. For example, adipose-derived MSC can be used. In embodiments, the stem cells may be autologous to the subject. If available, autologous stem cells can be beneficial to the subject because they will reduce or eliminate the potential for adverse immune responses, e.g., rejection of the stem cells or graft-versus-host disease. Autologous stem cells may be, e.g., stem cells isolated directly from the subject (e.g., MSC), or iPS cells produced from non-stem cells from the subject.

In some embodiments, in cases where autologous stem cells are not available or not indicated for a particular subject, allogeneic stem cells may be used. Allogeneic stem cells should be matched as closely as possible to the subject (e.g., via HLA genotype) in order to reduce the likelihood of rejection or graft-versus-host disease. In other embodiments, the stem cell donor is a first-degree-relative (e.g., parent, sibling, or child) of the subject, which increases the likelihood of finding a closely-matched donor. In yet other embodiments, the stem cell donor can be an extended relative of the subject. In some embodiments, the stem cell donor can be from the same race or ethnic group as the subject. However, certain stem cells can be immune-privileged and can be used allogeneically without matching between the donor and subject.

In yet another embodiment, methods for stimulating an immune response in a patient in need is described. In such embodiment, patients are administered effective amounts of a composition comprising, for example in the case of cancer, an inhibitor to inducible nitric oxide synthase, an inhibitor to indoleamine 2, 3-dioxygenase, a population of inducible nitric oxide synthase (iNOS)-deficient mesenchymal stem cells, a population of indoleamine 2,3-dioxygenase (IDO)-deficient mesenchymal stem cells or any combinations thereof. In a preferred embodiment, the method cause inhibition of the production of one or more of nitrogen oxide (NO), indoleamine 2, 3 dioxygenase (IDO), or prostaglandin E 2 (PGE2), 1-MT, 1400W, L-NMMA or other suitable agents. In this embodiment, the above mentioned inhibitors of iNOS or IDO are administered individually or as a mixture. In this aspect of the invention, the patient's status is post receiving a regimen of immune therapy including a regimen including the stimulated or modified MSCs described herein, or another immune therapy regimen which can include treatment with indicated interferons, antibody, cell therapy or other therapies that modulate immune response.

In embodiments, adipose-derived MSC are used for treatment of patients.

Appropriate MSC dosage can be, for example, 1×10³ cells, 2.5×10³ cells, 5×10³ cells, 1×10⁴ cells, 2.5×10⁴ cells, 5×10⁴ cells, 1×10⁵ cells, 2.5×10⁵ cells, 5×10⁵ cells, 1×10⁶ cells, 2.5×10⁶ cells, 5×10⁶ cells, 1×10⁷ cells, 2.5×10⁷ cells, 5×10⁷ cells, 1×10⁸ cells, 2.5×10⁸ cells, 5×10⁸ cells, 1×10⁹ cells, 2.5×10⁹ cells, 5×10⁹ cells, 1×10¹⁰ cells, 2.5×10¹⁰ cells, 5×10¹⁰ cells, 1×10¹¹ cells, 2.5×10¹¹ cells, 5×10¹¹ cells, 1×10¹² cells, 2.5×10¹² cells, 5×10¹² cells, 1×10¹³ cells, 2.5×10¹³ cells, 5×10¹³ cells, 1×10¹⁴ cells, 2.5×10¹⁴ cells, 5×10¹⁴ cells, 1×10¹⁵ cells, 2.5×10¹⁵ cells, 5×10¹⁵ cells, or more, or the like.

In embodiments, appropriate MSC dosage can be, for example, between 1×10³ cells and 2.5×10³ cells, between 5×10³ cells and 1×10⁴ cells, between 2.5×10⁴ cells and 5×10⁴ cells, between 1×10⁵ cells and 2.5×10⁵ cells, between 5×10⁵ cells and 1×10⁶ cells, between 2.5×10⁶ cells, between 5×10⁶ cells and 1×10⁷ cells, between 2.5×10⁷ cells and 5×10⁷ cells, between 1×10⁸ cells and 2.5×10⁸ cells, between 5×10⁸ cells and 1×10⁹ cells, between 2.5×10⁹ cells and 5×10⁹ cells, between 1×10¹⁰ cells and 2.5×10¹⁰ cells, between 5×10¹⁰ cells and 1×10¹¹ cells, between 2.5×10¹¹ cells and 5×10¹¹ cells, between 1×10¹² cells and 2.5×10¹² cells, between 5×10¹² cells and 1×10¹³ cells, between 2.5×10¹³ cells and 5×10¹³ cells, between 1×10¹⁴ cells and 2.5×10¹⁴ cells, between 5×10¹⁴ cells and 1×10¹⁵ cells, between 2.5×10¹⁵ cells and 5×10¹⁵ cells, or more, or the like.

In embodiments, appropriate MSC dosage can be, for example, not less than 1×10³ cells, not less than 2.5×10³ cells, not less than 5×10³ cells, not less than 1×10⁴ cells, not less than 2.5×10⁴ cells, not less than 5×10⁴ cells, not less than 1×10⁵ cells, not less than 2.5×10⁵ cells, not less than 5×10⁵ cells, not less than 1×10⁶ cells, not less than 2.5×10⁶ cells, not less than 5×10⁶ cells, not less than 1×10⁷ cells, not less than 2.5×10⁷ cells, not less than 5×10⁷ cells, not less than 1×10⁸ cells, not less than 2.5×10⁸ cells, not less than 5×10⁸ cells, not less than 1×10⁹ cells, not less than 2.5×10⁹ cells, not less than 5×10⁹ cells, not less than 1×10¹⁰ cells, not less than 2.5×10¹⁰ cells, not less than 5×10¹⁰ cells, not less than 1×10¹¹ cells, not less than 2.5×10¹¹ cells, not less than 5×10¹¹ cells, not less than 1×10¹² cells, not less than 2.5×10¹² cells, not less than 5×10¹² cells, not less than 1×10¹³ cells, not less than 2.5×10¹³ cells, not less than 5×10¹³ cells, not less than 1×10¹⁴ cells, not less than 2.5×10¹⁴ cells, not less than 5×10¹⁴ cells, not less than 1×10¹⁵ cells, not less than 2.5×10¹⁵ cells, not less than 5×10¹⁵ cells, or more, or the like.

In embodiments, appropriate MSC dosage can be, for example, not more than 1×10³ cells, not more than 2.5×10³ cells, not more than 5×10³ cells, not more than 1×10⁴ cells, not more than 2.5×10⁴ cells, not more than 5×10⁴ cells, not more than 1×10⁵ cells, not more than 2.5×10⁵ cells, not more than 5×10⁵ cells, not more than 1×10⁶ cells, not more than 2.5×10⁶ cells, not more than 5×10⁶ cells, not more than 1×10⁷ cells, not more than 2.5×10⁷ cells, not more than 5×10⁷ cells, not more than 1×10⁸ cells, not more than 2.5×10⁸ cells, not more than 5×10⁸ cells, not more than 1×10⁹ cells, not more than 2.5×10⁹ cells, not more than 5×10⁹ cells, not more than 1×10¹⁰ cells, not more than 2.5×10¹⁰ cells, not more than 5×10¹⁰ cells, not more than 1×10¹¹ cells, not more than 2.5×10¹¹ cells, not more than 5×10¹¹ cells, not more than 1×10¹² cells, not more than 2.5×10¹² cells, not more than 5×10¹² cells, not more than 1×10¹³ cells, not more than 2.5×10¹³ cells, not more than 5×10¹³ cells, not more than 1×10¹⁴ cells, not more than 2.5×10¹⁴ cells, not more than 5×10¹⁴ cells, not more than 1×10¹⁵ cells, not more than 2.5×10¹⁵ cells, not more than 5×10¹⁵ cells, or more, or the like.

The disclosed methods can also involve the co-administration of bioactive agents with the stem cells. By “co-administration” is meant administration before, concurrently with, e.g., in combination with bioactive agents in the same formulation or in separate formulations, or after administration of a therapeutic composition as described above.

Disclosed pharmaceutical compositions can also be provided as a kit. A kit of the invention can contain a pharmaceutically acceptable carrier; an isolated population of stimulated or modified MSC, and further instructions for using the kit in a method for attenuating an immune response. In this aspect of the invention, the cells stimulated with, for example, cytokine components of the kit can be administered. The kit also optionally may include a means of administering the cells, for example by injection. In an optional embodiment, the compositions of this invention suitable for parenteral administration can further contain antioxidant(s) in combination with one or more pharmaceutically-acceptable sterile isotonic aqueous or nonaqueous solutions, suspensions or in the form of sterile lyophilized powders which may be reconstituted into sterile injectable solutions or dispersions just prior to use, which may contain the combination of the antioxidants, minerals and vitamins, buffers, solutes which render the final formulation isotonic.

As used herein, the phrase, “bioactive agents” refers to any organic, inorganic, or living agent that is biologically active or relevant. For example, a bioactive agent can be a protein, a polypeptide, a nucleic acid, a polysaccharide (e.g., heparin), an oligosaccharide, a mono- or disaccharide, an organic compound, an organometallic compound, or an inorganic compound. It can include a living or senescent cell, bacterium, virus, or part thereof. It may include a biologically active molecule such as a hormone, a growth factor, a growth factor-producing virus, a growth factor inhibitor, a growth factor receptor, an anti-inflammatory agent, an antimetabolite, an integrin blocker, or a complete or partial functional sense or antisense gene, including siRNA. It can also include a man-made particle or material, which carries a biologically relevant or active material. An example is a nanoparticle comprising a core with a drug and a coating on the core.

Bioactive agents may also include drugs such as chemical or biological compounds that can have a therapeutic effect on a biological organism. Non-limiting examples include, but are not limited to, growth factors, anti-rejection agents, anti-inflammatory agents, anti-infective agents (e.g., antibiotics and antiviral agents), and analgesics and analgesic combinations. Anti-inflammatory agents may be useful as additional agents to counteract the inflammatory aspects of the fibrotic process.

Combinations, blends, or other preparations of any of the foregoing examples can be made and still be considered bioactive agents within the intended meaning herein. Aspects of the present disclosure directed toward bioactive agents may include any or all of the foregoing examples. In other embodiments, the bioactive agent may be a growth factor. A growth factor is any agent which promotes the proliferation, differentiation, and functionality of the implanted stem cell. Non-limiting examples of suitable growth factors may include, but are not limited to, leukemia inhibitory factor (LIF), epidermal growth factor (EGF), fibroblast growth factor (FGF), insulin-like growth factor (IGF), vascular endothelial growth factor (VEGF), human growth hormone (hGH), platelet-derived growth factor (PDGF), interleukins, cytokines, and/or combinations thereof.

In some embodiments, the bioactive agent can be an immunosuppressive agent. An immunosuppressive agent is any agent which prevents, delays the occurrence of, or decreases the intensity of the undesired immune response, e.g., rejection of a transplanted cell, tissue, or organ, or graft-versus-host disease. Preferred are immunosuppressive agents which suppress cell-mediated immune responses against cells identified by the immune system as non-self. Examples of immunosuppressive agents may include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art. In other embodiments, bioactive agents that may be administered include anti-fibrotic agents including, but not limited to, nintedanib, INT-767, emricasan, VBY-376, PF-04634817, EXC 001, GM-CT-01, GCS-100, Refanalin, SAR156597, tralokinumab, pomalidomide, STX-100, CC-930, simtuzumab, anti-miR-21, PRM-151, BOT191, palomid 529, IMD1041, serelaxin, PEG-relaxin, ANG-4011, FT011, pirfenidone, F351 (perfenidone derivative), THR-184, CCX-140, FG-3019, avosentan, GKT137831, PF-00489791, pentoxifylline, fresolimumab, and LY2382770.

Further disclosure related to isolation, stimulation, modification of stem cells, and their therapeutic uses, can be found, for example, in U.S. Pat. Nos. 8,685,728, 9,301,979, US20190046577A1, Genetic Engineering of Mesenchymal Stem Cells and Its Application in Human Disease Therapy. Hum Gene Therapy; 2010 November; 21 (11): 1513-1526, and Therapeutic Potential of Genetically Modified Mesenchymal Stem Cells; Gene Therapy volume 15, pages 711-715 (2008), each of which is incorporated by reference in its entirety.

### Example 1—Treatment of Atopic Dermatitis

MicroRNAs (miRNAs), which interfere with mRNA translation, are becoming recognized as powerful biomarkers for various diseases. Here, we examine miR-203 and miR-483 expression, along with the CD4⁺/CD8⁺ cell ratio, total IgE, expression levels of the three AD associated genes (PIAS1, RORA, SH2B1) and a panel of cytokines (IL-4, IL-10, IL-13, IL-31, IFN-γ, TGF-β1, TNF-α) in AD dogs compared to their healthy controls.

### Animals

A total of nine client-owned AD dogs (six males and three females) with naturally occurring non-seasonal AD were enrolled in this study from August 2017 to March 2018. The AD dog breeds reported by owners include Miniature Pinscher mix, Golden Retriever, Brittney Spaniel, German Shepard, Shih Tzu, Papillon, Great Dane, Cocker Spaniel, Boxer, Poodle and Terrier mix. In addition, another eight client-owned healthy dogs without AD (5 males and 4 females) were enrolled in this study as controls. The healthy dog breeds reported by owners include Rat Terrier, Chihuahua Mix, Chihuahua, Terrier Mix, Pitbull Mix, Plot Hound, and Cattle Dog Cross.

### Inclusion Criteria for AD Dogs

Clinical diagnosis of AD was based on detailed interpretation of patient history and clinical signs and exclusion of other possible skin dermatosis that can present as AD. Flea combing, skin scrapings, skin cytologies and elimination diet trials were performed. These are in accordance with the guidelines developed by the International Committee for Allergic Diseases in Animals (ICADA) diagnosis of canine AD. Patients in AD group were over one year of age, with a body condition score of at least 4 on a 9-point scale. Underlying systemic diseases were ruled out through thorough physical examination and serum chemistry and hematology analysis. Participants should be on effective flea control.

### Exclusion Criteria for AD Dogs

Clinical evidence of ectoparasite infestations (flea allergy dermatitis, scabies etc.), bacterial or fungal cutaneous infections, food allergies, and seasonality of the cutaneous condition resulted in exclusion from the study. Ongoing treatment with anti-inflammatory or immunosuppressive medications (antihistamines, glucocorticoids, and NSAIDS), also resulted in exclusion, unless appropriate weaning times were followed.

### Inclusion Criteria for Healthy Dogs

Participants that were more than 1 year of age, had a body condition score of at least 4 on a 9-point scale, with no history or clinical signs of pruritus or immune modulating disease conditions were enrolled in the study.

### Flow Cytometry

Peripheral Blood Mononuclear Cells (PBMCs) were isolated from whole blood collected in EDTA vacutainers. 2 mL of whole blood was then diluted with 6 mL of PBS. Diluted whole blood was layered on top of 2 mL of Ficoll-Paque PLUS (GE Healthcare Catalog #17-1440-02) and centrifuged at 2500 rpm for 25 minutes (no brake). The PBMC interphase was collected. Next, red blood cells (RBCs) were lysed with 1×RBC Lysis Buffer (BioLegend Catalog #420301) followed by spinning and resuspension in cell staining buffer (BioLegend Catalog #420201). Antibody staining was conducted using Bio-Rad Anti-dog CD3 Clone CA17.2A12: FITC, CD4 Clone YKIX302.9: RPE, CD8 YCATE55.9: Alexa Fluor647 (Bio-Rad) and staining with 10 μL of isotype control Bio-Rad MSE IgG1: FITC/RAT IgG2a: RPE/RAT IgG1: Alexa Fluor647 (Bio-Rad Catalog #TC023). Cells were resuspended in 400 ul of cell staining buffer, stained with 5 μL of 7-AAD Viability Dye (BioLegend Catalog #420404) and analyzed on the BD Accuri C6 Flow Cytometer.

### Serum ELISA Analysis

Serum ELISA analysis was carried out according to the manufacturer's protocols, and the following ELISA kits were used for this study.

| Canine | | | Catalog |
|---|---|---|---|
| Cytokine | Company | ELISA Kit | Number |
| IL-4 | NeoBioLab | Canine IL4 ELISA Kit | CI0014 |
| IL-10 | R&D | Canine IL10 Quantikine | CA1000 |
| | Systems | ELISA Kit | |
| IL-13 | NeoBioLab | Canine IL13 ELISA Kit | CI0043 |
| IL-31 | NeoBioLab | Canine IL31 ELISA Kit | CI0041 |
| IFN-γ | R&D | Canine IFN-γ Quantikine | CAIF00 |
| | Systems | ELISA Kit | |
| TGF-β1 | R&D | Mouse/Rat/Porcine/Canine | MB100B |
| | Systems | TGF-β1 Quantikine | |
| | | ELISA Kit | |
| IgE | Abcam | Canine IgE ELISA Kit | Ab157700 |
| TNF-α | R&D | Canine TNF-α Quantikine | CATA00 |
| | Systems | ELISA Kit | |

### RNA Extraction and Real-Time PCR

RNA was extracted from PBMCs by using the RNeasy Mini Kit (Qiagen, Catalog #74104) according to manufacturer's protocol with an additional DNAse I digestion step. Extracted RNA was then converted into cDNA using the High Capacity cDNA Reverse Transcription Kit following the manufacturer's instructions (Applied Biosystems, Catalog #4368814). The reactions were performed in triplicate on Bio-Rad CFX connected Real-Time PCR system. Canine glyceraldehyde-3-phosphate dehydrogenase (GAPDH) gene was used as an internal control. The following primer sets were used:

| Gene | Forward | Reverse |
|---|---|---|
| Canine PIAS1 | TGGAGTTGATGGATGCTTGAG | GGACACTGGAGATGCTTGAT |
| | (SEQ ID NO: 1) | (SEQ ID NO: 2) |
| Canine RORA | AAGGCTGCAAGGGCTTTTTC | CTGCGTACAAGCTGTCTCTT |
| | (SEQ ID NO: 3) | (SEQ ID NO: 4) |
| Canine SH2B1 | CGTCCTCACTTTCAACTTCCA | GACACGACATAGCTGACAAGA |
| | (SEQ ID NO: 5) | (SEQ ID NO: 6) |
| Canine GAPDH | GGAGAAAGCTGCCAAATATG | ACCAGGAAATGAGCTTGACA |
| | (SEQ DI NO: 7) | (SEQ ID NO: 8) |

### MicroRNA Expression by Real-Time PCR

Whole blood collected with EDTA-coated tubes was spun down and the plasma supernatant containing miRNA was collected. Extraction of miRNA was performed by following the protocol outlined in miRNeasy Serum/Plasma Kit (Qiagen, Catalog #217184) and the miRNeasy Serum/Plasma Spike-In Control was used (Qiagen, Catalog #219610). The reverse transcription was conducted by following the protocol of “Taqman Small RNA Assays” (Applied Biosystems, Catalog #4366596). TaqMan real-time PCR assays was performed Bio-Rad CFX connected Real-Time PCR system according to the manufacturer's instructions. Data were normalized to the internal control miR-39. The following TaqMan probe and primer sets (ThermoFisher) were used: miR-39 (RT 000200), miR-203 (RT 000507) and miR-483 (RT 002560).

### Results

### PDE4D Gene Expression is Significantly Upregulated in AD Dog PBMCs

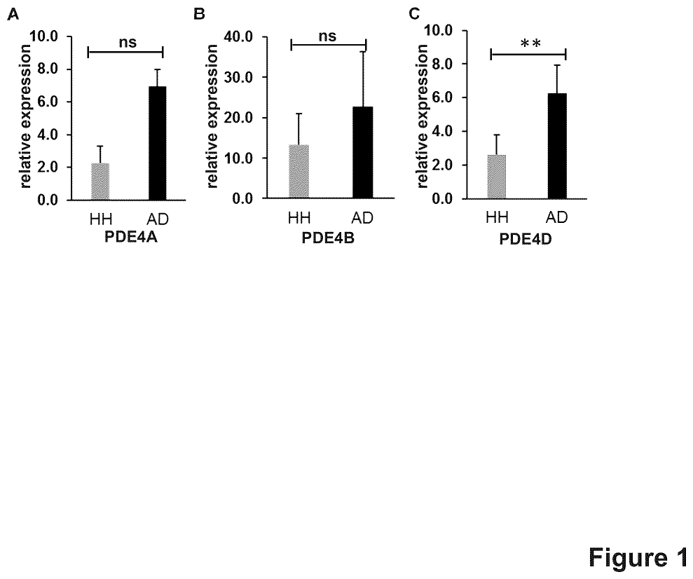

Phosphodiesterase 4 (PDE4) is a cyclic AMP-degrading enzyme in leukocytes. Several decades ago, increased PDE activity was demonstrated in patients with atopic dermatitis (AD). Currently, several PDE4 inhibitors in both topical and oral formulation have been developed to target the inflammatory cascade of AD. This review shows the pathogenic rationale behind these inhibitors, and discusses multiple PDE4 inhibitors that are under evaluation or in the market. PDE4 inhibitors may be considered as favorable agents in the repertoire of current interventions for AD. Multiple studies have shown that inhibition of PDE4 is beneficial to canine AD. However gene expression of PDE4s in PBMCs of canine AD has not been reported. We therefore examined all four PDE4 isoforms to verify whether any of these isoforms may be a potential marker(s) for canine AD. Blood samples were collected from eight healthy dogs and nine AD dogs, and RNA was then extracted from PBMCs of these blood samples. The RT-PCR results indicated that three out of four PED4A isoforms (PDE4A, PDE4B and PDE4D) are upregulated in the AD samples whereas PDE4C gene is not detectable in both AD and heathy dog samples (FIG. 1). Particularly, PDE4D gene expression in AD samples shows statistically significant upregulation by approximately 2.4-fold in comparison to that of the health control samples (p<0.01) (FIG. 1C). Though the gene expression levels of PDE4A and PDE4B were also elevated in AD samples verse the health control samples, both of the increases are not statistically significant (P=0.053 for PDE4A and P=0.097 for PDE4B). In summary, PDE4D may be a potential marker for AD dogs.

MIR-203 and miR-483 are Upregulated in AD Dog Plasma.

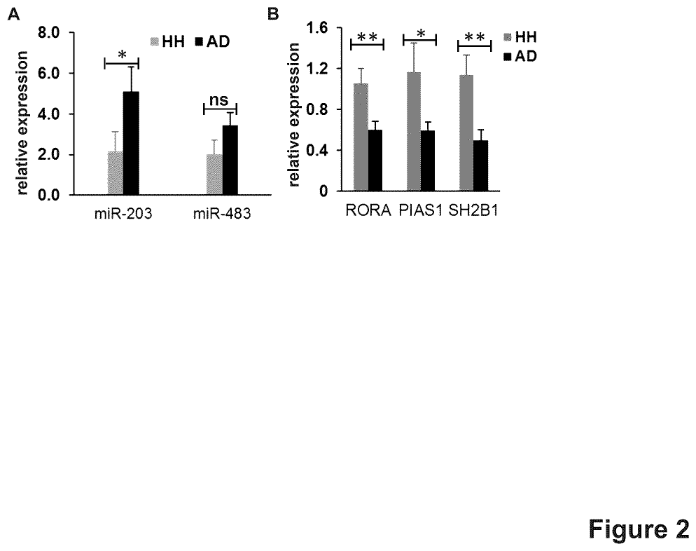

Circulating MIR-203 and miR-483 were previously shown to be upregulated in children AD sera. However, to date, no study of miRNAs in AD dogs has been reported. As dog AD has many similar characteristics with its human counterpart, we examined miR-203 and miR-483 expression changes in the plasma of both AD dogs and healthy controls. Blood samples were collected from eight healthy dogs and nine AD dogs, and their plasma was prepared, followed by miRNA extraction. RT-PCR reactions were conducted to quantify expression of miR-203 and miR-483, and miR-39 was used as the internal control. Our results showed elevated expression levels of miR-203 and miR-483 by approximately 2.5-fold (P=0.047) and 1.6-fold (statistically not significant P=0.074) respectively in the plasma of AD dogs in comparison with those of the healthy controls (FIG. 2A). This result suggests that miR-203 may be a possible biomarker for AD of both humans and dogs.

### Verification of PIAS1, RORA and SH2B1 Gene Downregulation in the PBMCs of AD Dogs

We performed RT-PCR reactions to examine these gene expressions (PIAS1, RORA and SH2B1) in PBMCs of both the AD dogs and healthy controls. In agreement with the previous report, our results confirmed that expression levels of all three gene (PIAS1, RORA and SH2B1) were significantly downregulated in the PBMCs of the AD dogs by approximately 1.5-fold for RORA gene (P=0.007) and 2-fold for both genes of PIAS1 (P=0.027) and SH2B1 (P=0.004) in comparison with the healthy dogs (FIG. 2B).

The CD4⁺/CD8⁺ Ratio of T Lymphocytes, Cytokines and Total IgE in AD Dogs

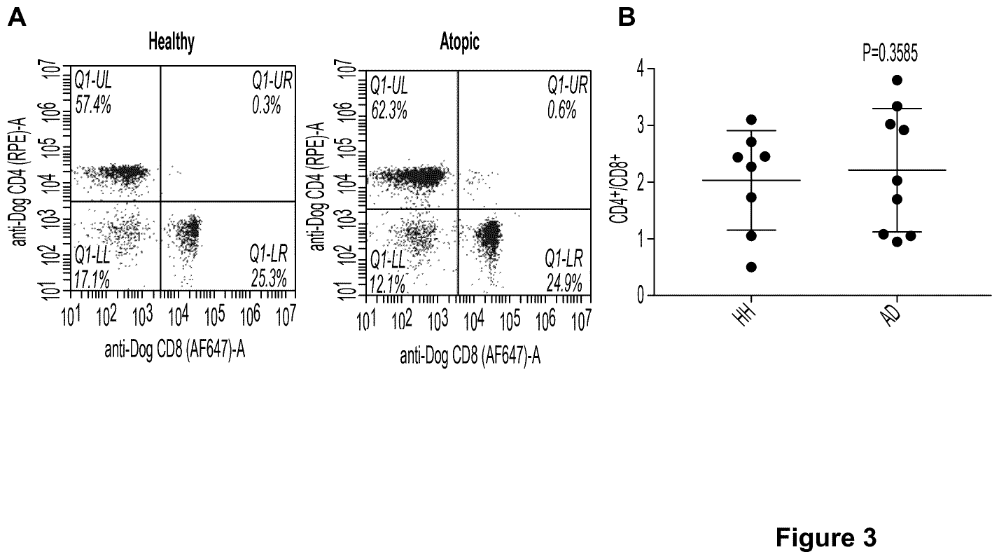

T lymphocytes are critical for the development and regulation of cell-mediated immune responses. We compared the CD4⁺/CD8⁺ ratio of T lymphocytes in the PBMCs from the AD dogs and healthy controls by flow cytometry analysis. Our data indicates a modest increase in CD4⁺ T Cells in the AD dogs' samples in comparison with the healthy controls (FIG. 3A), resulting in a slight elevation of the CD4⁺/CD8⁺ ratio in AD dogs (2.388±0.3747) vs. healthy controls (2.101±0.2826) (FIG. 3B). Nevertheless, this increase of CD4⁺/CD8⁺ ratio of T lymphocytes associated with canine AD was not statistically significant (P=0.3585).

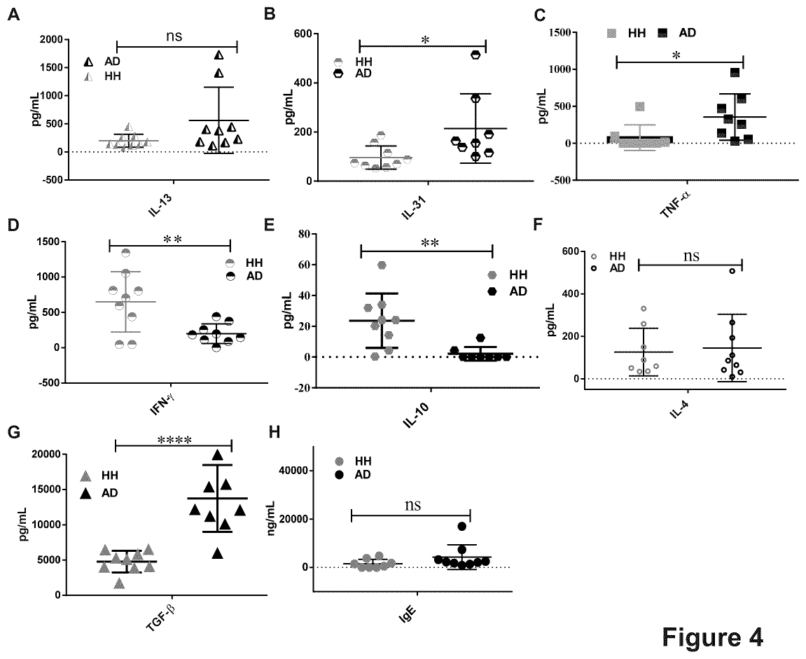

As canine AD is an inflammatory related-skin disease, we next examined a panel of cytokines of including TH2 cytokines (IL-4, IL-13 and IL-31), TH1 cytokine IFN-γ, anti-inflammatory cytokines (IL-10 and TGF-β1), and pro-inflammatory cytokine TNF-α by ELISA. Consistent with the previous reports, inflammatory cytokines IL-13, IL-31 and TNF-α were significantly elevated (FIGS. 4A, 4B and 4C statistically non-significant for IL-13) whereas the pro-inflammatory cytokine IFN-γ and anti-inflammatory cytokine IL-10 were dramatically decreased in AD patient sera (FIGS. 4D and 4E). In addition, expression of IL-4, which induces Th2 cell differentiation and B-cell class switching to IgE, was slightly increased in AD patient sera as compared to the healthy controls (FIG. 4F). This result is similar with the former report of IL-4 expression in canine AD patient plasma. In addition, previous reports about TGF-β1 expression in canine AD are controversial. For instance, Fedenko et al. showed a significant elevation of TGF-β1 in AD patient blood samples vs their healthy controls, whereas others reported opposite results. Our study indicated that TGF-β1 expression in the canine AD sera is elevated by approximately 2.8-fold in comparison with the healthy controls (FIG. 4G). Finally, the total serum IgE level displayed no significant difference in the sera of AD dogs and the healthy controls which is in agreement with previous reports (FIG. 4H).

### Discussion

Approximately 10% of dogs suffer from AD and the pathogenesis of canine AD has not been fully understood. To date, diagnosis of canine AD relies on a combination of patient history, clinical examination, allergy testing and response to diet trials/therapies, and reliable AD specific biomarkers are lacking. Here, we assessed all four PDE4 isoforms, specific miRNA expression, expression levels of genes associated with canine AD, the CD4+/CD8+ ratio of T lymphocytes, and a panel of cytokines in peripheral blood of both canine AD dogs and healthy controls. We, for the first time, report statistically significant expression increase of PDE4D gene in PBMCs and miR-203 in sera from AD dogs. Particularly, the increase of PDE4D gene expression is well aligned with previous studies of that inhibition of PDE4 is beneficial to both humans and dogs with AD. Moreover, the increase of miR-203 in plasma of dogs is consistent with the previous study in serum of children with AD, highlighting similarities of AD in both dogs and humans. In addition, controversial results of the CD4⁺/CD8⁺ T cell ratio were reported in association with AD dogs before, and our result suggests a slight but not statistically significant increase of the CD4⁺/CD8⁺ ratio in AD dogs, which is in line with Beccati et al who showed no significant differences in the ratio of healthy dogs, atopic dogs and atopic dogs treated with cyclosporin.

Furthermore, our study demonstrates the down-regulation of three genes (PIAS1, RORA, SH2B1) that are associated with canine AD in comparison to healthy dogs. Interestingly, RORA was recently reported to involve in transactivation of IL-10 promoter, and downregulation of RORA may contribute to the decrease of serum IL-10 in our study.

Finally, our cytokine profiling showed significantly elevated expression levels of Th2 inflammatory cytokines IL-13 and IL-31 and pro-inflammatory cytokine TNF-α, and dramatically decreased expression levels of TH1 cytokine IFN-γ and anti-inflammatory cytokine IL-10 in AD dogs. These results suggest that the Th2 response is increased and the Th1 response is decreased in the isolated PBMCs from AD dogs. Particularly, IL-31 (a TH2 cytokine) has attracted a lot of attention in recent years for its role in pruritus and atopic inflammation, and recent studies demonstrated serum IL-31 levels positively correlate with disease severity in both children and dogs with AD. Although total serum IgE levels are elevated in the humans with AD, they show no clinical relevance in the AD dogs in this study, which is in agreement with previous reports. In addition, controversial results regarding immunosuppressive cytokine TGF-β1 expression have been shown in canine AD. Here, our study indicated that TGF-β1 expression was dramatically elevated in AD dog sera in comparison with healthy controls, and the reported variations of TGF-β1 expression could be attributed to the varying degree of severity of inflammation and pruritus in AD patients. Moreover, TGF-β1 is usually immunosuppressive and can block inflammatory reactions, and elevated levels might indicate a feedback loop in reaction to the inflammatory response. In summary, our study of biomarkers in peripheral blood may provide important insight and groundwork for developing a less-invasive method for rapid diagnosis of AD disease and assessment of treatment efficacy.

### Supplemental Data

TABLE 1
Healthy Age, Sex, Breed, Spay/Neuter

| | Age (Year & | | | |
|---|---|---|---|---|
| Patient | Month) | Sex | Breed | Spay/Neuter |
| H1 | 9 Years | Female | Rat Terrier | Spay |
| H3 | 9 Years | Male | Chihuahua | Neuter |
| H4 | 2 Years & 5 | Female | Mixed | Spay |
| | Months | | | |
| H6 | 1 Year & 2 Months | Male | Pitbull Mix | Neuter |
| H7 | 1 Year & 8 Months | Female | Plott Hound | N/A |
| H8 | 1 Year & 8 Months | Male | Plott Hound | Neuter |
| H9 | 2 Years | Female | Cattle Dog | Spay |
| | | | Cross | |
| H10 | 2 Years | Male | Cattle Dog | Neuter |
| | | | Cross | |

TABLE 2
Atopic Age, Sex, Breed, Spay/Neuter

| | Age (Year & | | | Spay/ |
|---|---|---|---|---|
| Patient | Month) | Sex | Breed | Neuter |
| AD400 | 10 Years & 7 | Female | Golden Retriever | Spay |
| | months | | | |
| AD500 | 5 Years & 2 | Male | Miniature | Neuter |
| | Months | | Pinscher Mix | |
| AD900 | 3 Years & 7 | Female | German Shepard | Spay |
| | Months | | | |
| AD1000 | 6 Years & 8 | Male | Shih Tzu | Neuter |
| | Months | | | |
| AD1200 | 5 Years and 10 | Female | Great Dane | Spay |
| | Months | | | |
| AD1300 | 10 Years & 1 | Male | Cocker Spaniel | Neuter |
| | Month | | | |
| AD1400 | 7 Years & 1 Month | Male | Boxer | Neuter |
| AD1500 | 8 Years & 1 Month | Male | Poodle | Neuter |
| AD1600 | 5 Years & 7 | Male | Terrier Mix | Neuter |
| | Months | | | |

### Example 2—Treatment of Atopic Dermatitis

An 8 year old canine suffers from atopic dermatitis. To treat the animal, autologous MSC at a dose of 1×10⁷ cells are administered subcutaneously via injection. Within a week, the patient's symptoms decrease.

### Example 3—Treatment of Atopic Dermatitis

A 4 year old dog suffers from atopic dermatitis. Autologous MSCs are administered at a dose of 2.5×10⁵ cells via injection. Within a week, the patient's symptoms decrease.

### Example 4—Treatment of Atopic Dermatitis

A 13 year old dog suffers from atopic dermatitis. Allogeneic Adipose-derived MSCs are administered at a dose of 1×10⁷ cells via injection. Within two weeks, the patient's symptoms decrease.

### Example 5—Stimulation of MSC

MSC are treated with a cell signaling molecule to stimulate anti-inflammatory and immune modulatory cytokine production.

### Example 6—Modification of MSC

MSC are transformed with non-native DNA to produce a specific cytokine.

### Example 7—Chemoattractive Property of MSCs is Induced by Proinflammatory Cytokines

In several studies, effective immunosuppression by MSCs in vivo has been achieved with as few as one to five MSCs per million somatic cells and often endures for months, with complete cure of immune disorders in some instances. Considering that MSCs are immobile after settling in tissues, and that immunosuppression is mediated by NO, which acts only very locally near its source, this immunosuppressive effect is astonishing. It was contemplated that cytokine-induced MSCs might have a mechanism to attract immune cells to their vicinity, where the locally high concentrations of NO could act effectively on the target T cells. To explore this, co-cultures of MSCs and splenocytes were monitored over time under the microscope. Upon anti-CD3-stimulation, the splenocytes were observed to actively migrate toward the spindle-shaped MSCs. In contrast, no migration occurred in the absence of anti-CD3 stimulation. Since splenocytes have limited viability, the lack of locomotion toward MSCs in the absence of stimulation might be due to the poor health of these cells in vitro. To exclude this, activated-splenocyte-supernatant-primed MSCs were examined for their ability to attract A1.1 T hybridoma cells, which survive well even in the absence of IL-2. Under these conditions, time-lapse microvideography revealed brisk migration of T cells toward MSCs within 1.5 hours of co-culture initiation. Without priming of MSCs, however, there was no net movement of T cells toward the MSCs. Therefore, MSCs promote the migration of T cells only after MSCs having been exposed to proinflammatory cytokines.

To examine the role of various cytokines in enabling MSCs to attract T cells, MSCs were pretreated with various combinations of recombinant cytokines and the resultant migration of pre-activated T cells in co-cultures was observed. This analysis indicated that the same T cell cytokine pairs (i.e., IFN gamma and TNF alpha, IFN gamma and IL-1 alpha, or IFN gamma and IL-1 beta) that had induced the immunosuppressive function of MSCs also caused them to attract T cells. Likewise, using antibody neutralization of specific cytokines, it was found that migration toward MSCs was prevented by anti-IFN.gamma. alone, or by blocking TNF alpha, IL-1 alpha and IL-1 beta as a threesome, identical to their effects on activated-splenocyte-supernatant-induced MSC suppression of T cell proliferation. Therefore, the cytokine-induced immunosuppressive function of MSCs is likely to depend on the migration of lymphocytes into proximity with MSCs, where NO levels are highest.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for treating atopic dermatitis (AD) comprising:
administration of mesenchymal stem cells (MSC) to a patient in need thereof;
wherein said patient has been diagnosed with AD by determining the expression level of phosphodiesterase 4D (PDE4D) gene in peripheral blood mononuclear cells (PBMCs) and comparing said expression levels with those in a patient without AD, wherein increased expression levels indicate a patient suffering from AD.

2. The method of claim 1, wherein said MSC are obtained from adipose tissue, bone marrow, umbilical cord or placenta.

3. The method of claim 2, wherein said MSC are obtained from adipose tissue.

4. The method of claim 2, wherein said MSC are obtained from bone marrow.

5. The method of claim 2, wherein said MSC are obtained from placenta.

6. The method of claim 2, wherein said MSC are obtained from umbilical cord.

7. The method of claim 2, wherein said patient is a mammal.

8. The method of claim 7, wherein said patient is a canine.

9. The method of claim 7, wherein said patient is a human.

10. The method of claim 7, wherein said administration comprises at least one of subcutaneous, intra-articular, intra-lesional, intravenous, intra-peritoneal or intramuscular administration.

11. The method of claim 10, wherein said administration comprises subcutaneous administration.

12. The method of claim 10, wherein said administration comprises intra-articular administration.

13. The method of claim 10, wherein said administration comprises intra-lesional administration.

14. The method of claim 10, wherein said administration comprises intravenous administration.

15. The method of claim 10, wherein said administration comprises intra-peritoneal administration.

16. The method of claim 10, wherein said administration comprises intramuscular administration.

17. The method of claim 10, where MSCs are administered 1-10 times.

18. The method of claim 17, where MSCs are administered at intervals of 1-6 months.
